(19) United States
(12) Patent Application Publication
(10) Pub. No.: US 2023/0128139 A1
(43) Pub. Date: Apr. 27, 2023

(54) CIRCADIAN LIGHT-TRACKING ENHANCEMENT FOR MOBILE DEVICES

(71) Applicant: Blue Iris Labs, Fairfax, CA (US)

(72) Inventor: Erik Russell PAGE, Fairfax, CA (US)

(21) Appl. No.: 17/844,014

(22) Filed: Jun. 19, 2022

Related U.S. Application Data
(60) Provisional application No. 63/271,138, filed on Oct. 23, 2021.

(51) Int. Cl. A61B 5/00; G01J 3/28

(52) U.S. Cl. CPC A61B 5/4857; A61B 5/0022; A61B 5/486; G01J 3/28; A61B 2560/0242

(57) ABSTRACT

Circadian health recommendations and/or automation instructions and spectral data on which they are based can be provided to a user through a mobile device that lacks a spectral sensor. A user mobile device lacking a spectral data uploads place and time data to a cloud-based circadian health system. A light-exposure model of the circadian health system estimates spectral data values based on the place and time data. The light-exposure model’s estimation can be based on data received by the circadian health system based on user devices equipped with spectral sensors and from other sources. A relatively small number of mobile user devices (with spectral sensors) can thus provide for spectral value estimates for a relatively large population of mobile user devices that lack spectral sensors—greatly expanding the range and number of people that benefit from improved circadian health.

### BACKGROUND

Circadian rhythms are physical, mental, and behavioral changes that follow a 24-hour cycle. These natural processes respond primarily to light and dark, secondarily to light spectra, and affect most living things, including humans. Circadian rhythms are regulated by biological clocks composed of specific molecules (proteins) that interact with cells throughout the body. Nearly every tissue and organ contains biological clocks. A master clock in the brain coordinates all the biological clocks in a living thing, keeping the clocks in sync. In vertebrate animals, including humans, the master clock is a group of about 20,000 nerve cells (neurons) that form a structure called the suprachiasmatic nucleus, or SCN. The SCN is in a part of the brain called the hypothalamus and receives direct input from the eyes.

Many people notice the effect of circadian rhythms on their sleep patterns. The SCN controls the production of melatonin, a hormone that makes you sleepy. It receives information about incoming light from the optic nerves, which relay information from the eyes to the brain. When there is less light—for example, at night—the SCN tells the brain to make more melatonin, making a person drowsy. In addition to affecting sleep, circadian rhythms have substantial effects on hormone release, eating habits and digestion, and body temperature.

Innovations such as artificial lighting, regulated room temperature control and highspeed travel have freed people from natural environmental rhythms at a cost of impaired health due to their circadian rhythms being out of synchrony with their artificial environments. Circadian interventions include increased time outside in daylight and enhancements to indoor lighting to mimic the brightness and spectral variations of daylight, and the blocking of artificial light at night, e.g., using eye covers.

While circadian interventions have effectively improved the health of certain people, for example, people afflicted by Alzheimer’s disease and related disorders (ADRD), it is believed many more people could be helped if their circadian exposure were monitored so it could be supplemented when low. However, to benefit the population at large, this tracking must be done without significantly affecting their lifestyles.

### BRIEF DESCRIPTION OF THE DRAWINGS

FIG. 1 is a schematic illustration of a cloud-based circadian health tracking system accessed through mobile devices.

FIG. 2 is a more detailed schematic illustration of the cloud-based circadian services system of FIG. 1.

FIG. 3 is a schematic of a computer system for implementing a circadian tracking enhancement process in the context of the system of FIG. 1 and other systems.

### DETAILED DESCRIPTION

The present invention provides a circadian health management system with enhancement for circadian tracking data by mobile devices. A user device set submits data including, at a minimum, time and place data, to a light-exposure model that outputs enhanced circadian data including brightness (e.g., measured as photopic luminance or melanoptic-equivalent luminance) and spectral data. The light-exposure model can be developed using data submitted by user devices that provide detailed circadian data (including brightness and spectral data). This invention provides users of devices that lack spectral sensors and even lack brightness sensors with spectral data and the circadian health benefits that flow from having such data available.

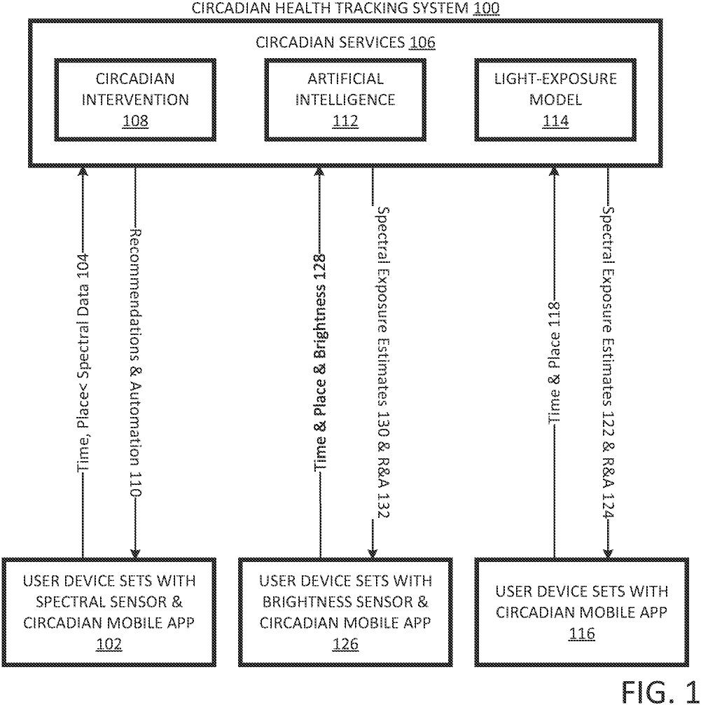

A circadian health tracking system 100, shown in FIG. 1, includes user device sets 102. Each user device set 102 includes a spectral sensor that tracks exposure to each of several frequency bands in the visible light range. Some embodiments include sensors that track light in the near infrared and/or near ultraviolet frequency ranges. Each device set 102 includes one or more devices worn or otherwise carried by a user. For example, a device set can be a smart phone including a spectral sensor, a smartphone and a separate spectral sensor that transfers data to a smartphone, or a standalone spectral sensor that can transfer data directly, e.g., to and from circadian services in the cloud. Since the pathway for light impacting circadian health is primarily through the eyes, the spectral sensor can be mounted in glasses, even contact lenses, or other head worn ornamentation or gear. Alternatively, spectral sensors can be integrated into watches, bracelets, rings, etc.

The spectral data is time-and-place stamped either by the spectral sensor and/or other device in the set. For example, a head-worn spectral sensor can provide time-stamped spectral data to a smartphone; the smartphone can then synchronize the time-stamped spectra data it receives with time-stamped place data that it collected using geosynchronous positioning system (GPS) data. The resulting time, place, and spectral data 104 can be uploaded to a cloud-based circadian service106. In an alternative embodiment, time data is not sent, but the place and other data that is sent is time-stamped upon reception.

Circadian services 106 includes a circadian intervention module 108 that can make recommendations and automations 110 based on received circadian, e.g., spectral, data. Recommendations can include, for example, spending more time outside in daylight, wearing eye covers at night, and adjusting spectra of circadian lighting. A circadian mobile app on a user device set 102 can be programmed to automate some interventions such as adjusting the color spectrum of a circadian lamp based on instructions from circadian intervention module 108.

Circadian services 106 includes an artificial intelligence (AI) module 112 that updates a light-exposure model 114 based on data received from user device sets 102. Light-exposure model 114 is used to make spectral exposure estimates for user device sets 116. Each user device set 116 uploads time and place data 118 to circadian services 106, but does not upload luminance or spectral data, e.g., because it lacks both a brightness and a spectral sensor.

Light-exposure model 114 returns spectral exposure estimates based on the time and place data 118. For a simple example, if the time and place data received from a device set of sets 116 matches that received from a device of sets 102, then the corresponding spectral data received from the sets 102 device can be returned to the sets 116 device. For a more complex model example, AI engine 112 can identify patterns and trends in spectral time, place, and data 104 that can be used by light-exposure model 114 to make spectral estimates without relying on contemporaneous data from device sets 102. In addition, spectral exposure estimates can be provided to circadian intervention module 108 so that recommendations and automation 124 can be provided to user sets 116 (which lack spectral sensors) just as they are provided to user sets 102 (which include spectral sensors), greatly expanding the population that can benefit from circadian health expertise.

User device sets 126 represent a case intermediate between spectral-sensor equipped capable device sets 102 and non-spectral-sensor equipped device sets 116. Device set of sets 126 can include a brightness sensor so that it can provide time, place, and brightness data 128 to circadian services 106. Light-exposure model 114 can use the extra brightness data to make spectral exposure estimates 130 with greater accuracy and confidence. Circadian intervention module 108 can use these better estimates to make more effective recommendations and automations 132. In addition, artificial intelligence 112 can use the brightness data to update light-exposure model 114 wherever spectral data may be lacking.

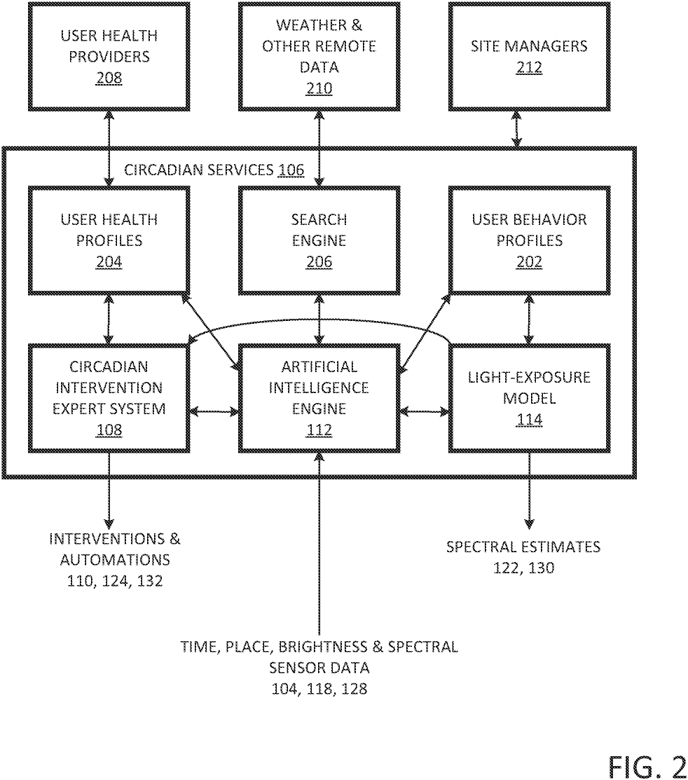

As shown in FIG. 2, circadian services 106 also include user behavior profiles 202, user health profiles 204, and a search engine 206. User behavior profiles 202 can include information to the effect one user works in an office eight hours a day five days a week and another is a landscaper who works outside most of the time. Such user-specific information can be used by light-exposure model 114 to improve spectral data estimates, especially in cases where place data is incomplete. User behavior profiles can be input by a user via AI engine 112.

User health profiles 204 include user-specific information such as responses to possible interventions. Individuals vary considerably in their needs and responses to circadian interventions; accordingly, the circadian intervention expert system can use this information, to the extent available, to tailor recommendations and automations. User heath profile data can be uploaded by the user or (with permission) the user’s health provider 208.

Artificial intelligence engine 112 can use search engine 206 to access weather and other remote data 210. AI engine 112 can provide data to light-exposure model 114 so that a cloudy weather forecast can be taken into account when determining spectral estimates. Similarly, poor air quality, tornado warnings, and the like can be used to prompt circadian intervention expert system 108 to recommend something other than a walk outside. Non-weather data of interest can include holiday schedules and pandemic lockdowns. Site managers, both human and automated can update and supplement circadian services components, including artificial intelligence engine 112 and circadian intervention expert system 108.

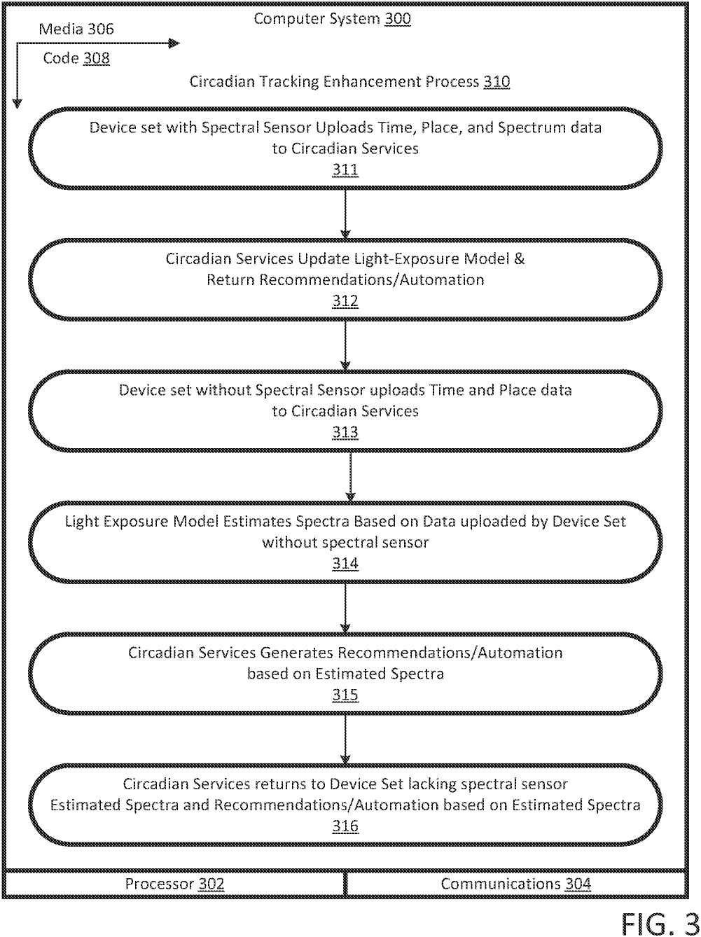

A computer system 300, shown in FIG. 3, includes the hardware for circadian health tracking system 100. Computer system 300 includes a processor 302, communications devices 304, and media 306. Media 306 is encoded with code 308 that, when executed by processor 302, implements a circadian tracking enhancement process 310. At 311, a user device sets with a spectral sensor uploads time, place, and spectral data to cloud-based circadian services 106 (FIG. 1). At 312, circadian services update a light-exposure model and returns recommendation and automation instructions.

At 313, a device set without a spectral sensor uploads time and place data to circadian services. In response to the time and place data, a light-exposure model provides spectral data estimates at 314. At 315, circadian services generates recommendations and automation instructions based on the estimated spectral data values. At 316, circadian services returns to the device set (that lacks a spectral sensor) estimated spectra, recommendations, and automation instructions based on the estimated spectra.

What do we even mean by “light exposure measurement”? How are these measurements made? Why is it important? And what can you learn from it? I’ll close by discussing something that I’ve been calling the Lighting Genome Project. Here I will discuss some of the significant societal and commercial benefits that could be realized if we had a better understanding of the light exposure people experienced today.

I pulled this figure out of this fantastic review of light and health research that was published in IES Journal Leukos last year. The citation is: Vetter et al, “A Review of Human Physiological Responses to Light: Implications for the Development of Integrative Lighting Solutions”, Leukos, The Journal of the Illuminating Engineering Society, 2021. For those interested in digging a little deeper, this paper not only gives a great scientific overview of this subject, but it also includes 8 pages of references, so no matter what your specific area of interest is here, you are should be able find a few rabbit holes to go down.

For those who aren’t going read it though, the oversimplified but unsurprising take away is this: “bright days and dark nights are good for human health.” So while I like this figure because it shows in one “simple” slide how light impacts our physiology it also is also useful for defining what we mean by light exposure measurement. When I am talking about light exposure measurement, I’m talking about measuring the physical properties of light that we know impact human physiology - which the authors of this report have called mediating factors. In case you haven’t already decoded it, this figure is showing how the physical properties of light are processed by our eyes, brain and other biological systems. These 5 characteristics of the light: timing, intensity, wavelength, duration, and light history link the physics of light (over here) to our biological response to it. (over here).

It’s also important to note that when you are doing spot measurements with a “normal” light meter, you are getting at best 2 of these 5 parameters. But the time factor is key when we are talking about light exposure and the impact on health. You really need to know when and for how long exposure is happening.

From a measurement perspective, if you are interested in correlating the light people experience to their health outcomes you would want to measure: ) Intensity, or how bright the light is, 2) Spectrum, or the radiant power of the light as a function of wavelength, timing - the time of day the exposure, duration, how long the exposure occurs, and light history, or looking beyond just the timing and duration of light exposure in current circadian phase.

What do we need measure to collect this information? First, we want to collect spectral power distribution (SPD). That is, we want to measure the physics of light, not the physics plus biological weighting factors that might be different for different people or be based on models that might be updated as our understand of the biological impacts of light grows. Second, we’d want to measure SPD continuously. Timing, duration and history of light exposure all matter, probably in ways we don’t fulling understand. And finally, we want to measure it at or near the eye. Sensors on the ceiling are not always a great proxy for light hitting the eye.

We built a tiny, battery powered, cloud-connected spectrometer to do this, which I’ll described in more detail below.

Why would you want to measure light exposure? From a big picture perspective, it’s pretty simple: light impacts human health. I hope at this point this is no longer a surprising or controversial statement to anyone here. This isn’t some woo-woo pseudo-science or some passing lighting industry marketing gimmick. This is factual statement based on a broad and growing collection of clinical trials being conducted by researchers around the world.

Yet, at the same time, we have very little information about the light exposure that humans experience. We believe that filling in this information gap will lead directly to improvements in health for people. I’ll go into more details on how later in the presentation when I talk about the Lighting Genome Project.

This quote captures the importance of measurement well: “Measurement is the first step that leads to control and eventually to improvement. If you can’t measure something, you can’t understand it. If you can’t understand it, you can’t control it.

First, it is important to remember that the intensity of light that we experience changes by 6 orders of magnitude each day. I’m showing this here on a log scale from moon light at less than 1 lux to direct sunlight, which can exceed 100,000 lux. Our eyes and visual systems can cover this huge dynamic range, but our ability to accurately perceive changes over this range is limited. This is based on a number of factors, including that, without our conscience awareness, our pupils are constantly adjusting to let more or less light into our eye based on the brightness of the environment we are in.

Now let’s compare how the modern lighting diet looks verses what I call the “paleo lighting diet,” or the diet that our pre-modern ancestors would have experienced. Again, we don’t really have light exposure data for typical modern humans let along pre-historic humans, so I’ve taken a swag here, assuming that the typical modern human is in indoors during most of the day, has electric lights on at night, and has little glowing LEDs on things in their bedroom. Meanwhile, the Paleo humans are typically outside in daylight during the day and in darkness at night.

The point I wanted to make here is this: scientists have now explained in great detail how over eons, we evolved extremely sophisticated and specialized biological systems that use our eyes to provide information to our nervous and endocrine systems that in turn take actions in response to cyclical changes to light stimulus in our environment. We lived this way for millennia, and then in a period of time that is meaningless from an evolutionary timescale, we have replaced this shared cyclical stimulus with to something very different.

So in this context, it doesn’t surprise me at all that study after study have shown that bright days and dark nights are good for us. Given what we now know about how light stimulus is detected and processed by our bodies, it would be much more shocking of these studies found anything else.

It is also worth noting that there is probably a lot more uncertainty in my estimate of what a “typical” modern person experiences here than what a typical human experienced a 100,000 years ago, which is a bit wild to think about. All of humanity used to live under bright days and dark nights -every day and every night for their whole life. Now, this species shared environmental input has likely been replaced with a wide range of experiences that vary between individuals and vary day to day for individuals.

So this here is where I think a lot of interesting things can be learned. What does the typical or average modern lighting diet look like? Is my swag close? How much variability is there between different populations of people? Are some populations seeing a more “paleo” type of lighting diet than others and does this have measurable impacts on sleep and related health outcomes? How much variability do individuals experience and how do these personal variables impact personal health outcomes? And perhaps most importantly, are their lighting interventions or automations that can help nudge individuals or populations towards light exposure patterns that result in measurable improvements in sleep or other health outcomes?

I also want to highlight the wide individual variability in how sensitive our circadian systems are to light. This recent study looked at dim-light melatonin offset - or how much evening light was required to suppress individuals’ melatonin production and found that some of the most sensitive subjects were significantly impacted by light levels under 10 lux while the least sensitive would need more than 300 lux to have a similar circadian impact. So if you REALLY wanted to know how your body is impacted by light exposure, you might want to both know what your actual light exposure is as well as your personal circadian sensitivity to light.

Finally, while it is useful know qualitatively that bright days and dark nights are good for us and that we should try to experience and design lighting systems to do this, there are certain things that you just need quantitative data for. This of course includes things like clinical trials or any situation where you are trying to correlate measurable light inputs to measurable health outcomes. Quantitative data is of course useful for AI, machine learning and big data tools that can help us find and understand these correlations. These data can also be used as inputs for intelligent building systems, which of course are only going to get more intelligent and sophisticated in the future. And finally, to be responsive to individual people and their individual lighting needs — rather than to the “average” needs or even worse “lowest common denominator” needs — we need data on these individuals.

So that first point — the need for quantitative data for clinical trials — is how we got to be involved in it this. As part of our NIH project, we wanted to be able to track all 5 of those mediating factors in real-world applications - which for us meant in Alzheimer’s cares care facilities. We ended up making our own small battery powered, cloud connected spectrometer for this, which we call the “Speck”. It can measure and store SPD and uploads data to the cloud where calculations of other lighting metrics — such as lux, CCT, mEDI, and CS — are made.

Now that we can gather SPD data, 24/7 from systems that can scale easily, we can do studies with larger sample sizes and that last longer. All this gives us a richer and more nuanced understanding of the impacts of light - including lighting interventions -on these populations.

Here I’m showing one SPD measurement taken by the Speck along with time-series circadian stimulus results from a week of wearable data and month of data from Specks fixed to the walls at our Alzheimer’s clinical trial site.

We are now selling these little spectrometers to others who are using them in clinical trials from NICUs to schools and offices to care facilities to gather data on how light exposure impacts specific health outcomes for specific populations.

Here is our requisite system architecture slide. The main thing I want to note here is that our system currently uses an IP-based protocol called Thread and gateway get our data from Specks to the cloud rather than something like Bluetooth directly from a sensor to a smartphone. When we move to more consumer oriented light exposure tracking, we are likely to add a Speck-to-phone link, but for now, we are focusing on gathering data in clinical trials and on enabling smart buildings applications. In these applications, combination of low-power use, reliability, range, and security have made Thread a great option for us.

So what can we learn from these data? To give you a taste of this, I’m going to start by quickly showing some examples of some of the ways I’ve been using the Speck.

After we started making the Specks in some volume a few months ago...I started wearing one around, whether that is hanging out in a hammock with my son JJ, going to the grocery store, or going on a sunset mountain bike ride.

I’ve also installed them in a number of places where I spend a lot of type, like this one behind my head in my office. Here I am showing how much light my very well daylit, east facing office gets, how it varies throughout the day and between days.

We’ve used them to look at how data from wearable sensors correlates to fixed location sensors. Put another way, how the light exposure a person experiences correlates with the places that person goes.

It’s not an entirely surprising result, but in these plots you can start to get a sense for how much more variable the light exposure is on a person than in a space. Most of these big spikes are periods when I left my office and was outside for a little bit. But you can also see that there are periods — specifically when I was sitting at my desk for a long time - probably working on this presentation — where the data trend together pretty nicely, indicating that in some situations, you might be able to estimate the light exposure people experience in a space with sensors in specific areas within buildings rather than needing to have sensors on people.

We’ve put Specks on both sides of windows to measure how light spectrum and intensity vary throughout the day and how this variance is impacted by window coatings.

And we’ve looked very closely at the light we are exposed to at night. It has been particularly shocking for me to see how much higher these light levels can be vs current scientific recommendations. On this graph, the red line represents the current recommendations of no more than 10 mEDI in the 3 hours before bed, along with my readings.

This peak here is from being outside just before sunset, which occurred around at 8pm on the day these data were collected.

Personally, I can say just being aware of my exposure has changed my behavior. I have been much more carefully about the amount of electric light I get after the sun goes down, even going so far as to brush my teeth without the lights on and listening to audiobooks in bed before sleep instead of reading.

Finally, we’ve also been looking directly at how sleep data is impacted by light exposure. When we are able to show, at a population level, how “better light exposure” can lead to “better sleep,” it will be a big deal. Here I’m showing my sleep tracker data and my light exposure data along with a simulation of how, with enough datapoints, we could establish correlations between the two. Can we show — for individuals and populations —that better light scores lead to better sleep scores?

Light scores?? What’s that?! I personally believe that - at least for consumer facing products and probably for anything that is used by people who aren’t lighting nerds, we will eventually end up with a light exposure “score” that provides information on how well you are doing in getting your bright day and dark night. Similar to the sleep scores that have been developed by companies that sell sleep trackers, light scores could provide a single numerical value that attempts to quantify how closely light exposure matches a target exposure. Ideally these numbers would be science-based, establishing exposure targets that are based on our latest understanding of what exposure profiles are shown to be most beneficial to health and wellness. Even better if these exposure targets are personalized - including considerations for the individuals target wake and sleep times. Better still if they include feedback from their sleep tracker or information on the individuals particular sensitivity to light exposure. And while these light score numbers can and should have a lot going on under the hood, they should be very easy for the layperson to understand and take action as needed. For example, if they get a low light score, they might be able to tap the score for more details where they might see that they had a pretty good “bright day” but that they also had a pretty “bright night” too, which is why their score suffered.

I believe that there is a place for light exposure scores at the heart of fitness and health trackers moving forward. Our phones and watches tell us our steps now not because it is more important that our light exposure, but because it is easier to measure.

I coined the phrase “Lighting Genome Project” as a nod to the Human Genome project, which was the largest collaborative biological scientific research project in history. While every human has a unique genome, the vast majority of the genome is shared across all humans. The project to map the human genome helped us understand everything from genetic risks associated with specific diseases to the function that specific genes have and what happens when there are mutations in these genes. Ultimately, this project has produced enormous benefits societally, commercially, and lives of real people.

The Lighting Genome Project is a vision of a scientific research project with the goal of mapping the light exposure profiles that humans experience. While each individual experiences their own light exposure profile, or their own personal “lighting exposure genome” the vast majority of us are likely to experience darker days and brighter nights that our ancestors. Having a better population level mapping of the “light exposure genome” will allow us to see how different light exposure profiles correlate to specific human health outcomes. And knowing even more about an individual’s lighting genome —both the light exposure they experience and their sensitivity to that exposure — could have significant health implications for the individual.

This analogy isn’t perfect and I fully acknowledge the ridiculousness of comparing this kernel of an idea with one of the most successful scientific efforts in human history. But carrying this analogy a little further, we can think of the modern lighting diet is a “mutation” on the human lighting genome and we are living the results. So far initial data suggests the mutation is having a significant negative impact in a broad number of areas, but more data is needed! And just as the Human Genome Project provided vast social, commercial and personal benefits, the Lighting Genome Project offer similar benefits.

Another way to frame it is this: given that we know that light exposure impacts health and that most people probably need brighter days and darker nights that they are currently getting: what are the implications for public health and for the lighting industry? What impact could it have if we were able to quantify what we meant by “bright days” and “dark nights”? Or if we could quantify how much better our health outcomes could be or what populations are likely to see the most significant benefits. Again, it comes back to this graph I shown previously. For eons we experience this and evolved complicated biological systems that used this signal to tell our bodies that it was time to do sometime. Then, essentially overnight, we changed that signal to something that looks very different. And as my favorite 80’s band stated, “you cannot go against nature, because when you do go against nature, that is part of nature too.” We can go against natures circadian cycles, but when we do, we should not be surprised when has an impact on our natural biological systems.

So what are some of the potential public health and lighting industry benefits associated with better understanding light exposure? First, let’s consider individuals or consumers. It’s easy to imagine wearables and apps that monitor individuals light exposure and encourage them to get the right type of light at the right time. Ideally these systems would give them feedback or “proof” showing them that when they follow the recommendations or using automations, they see measurable improvements in specific health-related metrics, such as their sleep. And better still of these light exposure targets are personalized for the individual, based on their environment, chronobiology and schedule.

Tools like this should help the lighting industry demonstrate to customers that lighting — and light and health products specifically — have benefits beyond simply allowing them to see in an otherwise dark room. Whether a company is selling sensors and app or lights, luminaires, or control systems that use these data, there are new opportunities to develop products that can prove their value to users quantitively.

The healthcare industry presents some real opportunities. Quantitative data on the light exposure patients experience that can be merge with data on their personal health metrics can open new doors to using lighting as a nonpharmacological intervention. This is what Mariana and others have been doing successfully in clinical trials, but these approaches need to be “productized” and made understandable, useful, and actionable for patients and care providers. We’ve already discussed how big this market is. And while there is no doubt that much red tape will need to be cut before we get there, lighting input data along with health metric output data should help us gather enough “proof” the efficacy of lighting solutions as nonpharmacological interventions to ultimately support things like insurance and Medicare reimbursements for lighting-based therapeutics in specific healthcare applications.

Thinking about commercial buildings more generally - quantitative data on light exposure can be used for a number of purposes from verifying that occupied spaces meet emerging light and health standards to looking for correlations between measurable outputs — such as sicks days and productivity — to lighting exposure inputs. I believe there are huge opportunities here to develop new lighting products that rely in a richer set up light exposure inputs to provide lighting solutions that help to improve the lighting diets of the people in these spaces. This may be especially true in more sophisticated smart buildings that include things like people tracking systems. For example, if building systems can know both where you are and what the lighting conditions are at that location, an estimate of your light exposure while you are in that building could be made without the need of a wearable.

On potential benefit in this application would be the ability to quantify the 3-30-300 argument. This is the rule of thumb - which probably is long overdue for an inflation adjustment - posits that we spend $3/sf for the utilities in a space, $30/sf for space itself, and $300/sf for the people in the space. The argument of course is that, in buildings, you should prioritize investments that make people more efficient because even small improvements here are likely to payback quicker than large improvements in space utilization or energy use. Up to this point, the weak link in this argument has been the “proof” of this impact. While the current version of the Speck does not measure “worker productivity” it does allow us to quantify light exposure in buildings, which can be compared to measurable outputs, such as test scores, healthcare costs, or specific productivity metrics.

So, beyond the societal and commercial opportunities that we’ve discussed so far, there is another aspect to the healthcare related opportunities for lighting that shouldn’t be overlooked. And it is how incredible personal and personally motivating things can be here

I want to do everything I can — for my son and in memory of my mom — to help reduce the suffering and improved the health of people in similar situations because I have seen it up close and can empathize. And of course, everyone in this room, and everyone you will be selling “healthy lighting” to has their own personal stories about their health and the health of people they love. I bring all this up in part because this is something of a paradigm shift for the lighting industry. Yes, I am also motivated to help develop lighting systems that are efficient, have good color, and dim well, but, I’m sorry, it’s not the same as developing systems that can help make a sick person become well.

So, here are the questions that I want to leave you with regarding the Lighting Genome Project. Are there common interests in the lighting industry in developing real-world scientific evidence of the impact of light exposure on human health? If there are, can we accelerate this process — for the benefit of society as well as for the commercial benefit of the lighting industry — by collaborating? This could range from an effort as modest as developing tools to make light exposure data easier to something as ambitious a broad coalition representing the $30 billion US lighting industry requesting $10’s of millions from US government to quantify impact of the “bright day, dark night effect” and the associated financial impactions for $4 trillion healthcare system.

The industry has been looking for the next value-add beyond just higher efficacy, longer lifetimes, and better dimming. We talk about Human Centric Lighting, but only loosely deliver it because there hasn’t been a cohesive approach to quantify lighting exposure and follow through with recommendations that our lights can implement.

Business Overview: Light exposure (spectrum, intensity, duration, timing, and history) impacts human health (and nearly all living things on Earth) in many scientifically documented ways. Yet it is difficult for individuals, researchers, or institutions to accurately monitor light exposure in order to improve individuals/groups/populations. Blue Iris Labs is focusing on developing tools to allow individuals/institutions monitor and control (manually or automatically) light exposure to improve health outcomes.

Personal light exposure trackers: products, systems, and ecosystems that allow for the measurement or estimation of an individuals daily light exposure. Generally speaking, these systems are “data gathering” approaches that measure/estimate light exposure and provide this information to users in ways that are useful/actionable (e.g. primary data on exposure for researchers; actionable recommendations for individuals etc.). There are a wide range of products/services/systems that may fall into this category including the current Speck device

More sophisticated light exposure trackers that measure a wider spectral range, have better accuracy and/or resolution, and/or are located at or closer to the eye (e.g glasses, contact lenses). In some cases, increases in accurate/resolution etc would be achieved by simply using higher procession hardware (e.g. higher resolution spectral chips). In other cases, measurement improvements would come from data from other inputs (e.g. camera images, GPS locations, indoor people tracking systems, etc) or from knowledge associated with our other products in our ecosystem (e.g. lower accuracy devices can be ground-truthed against higher accuracy devices when in close proximity; larger deployments of lower accuracy devices that also include GPS data could be used along with smaller deployments of higher accuracy devices to achieve insights that would not be possible with either device alone; corrections to measurements made at locations other than near the eye (e.g. wrist, chest) could be made based on correlations between measurements made at these locations and those made at/near the eye). These applications will initially be targeted for researchers who would be willing to pay more for systems that more accurately estimate light hitting the retina.

Less expensive light trackers that target the consumer market. Moving in the other direction from our current Speck, there may be products/services we provide that prioritize either lower-cost hardware systems (e.g. lux sensor only) or no-hardware systems (e.g. app on smart phone or watch or lens) in order to achieve a lower “consumer friendly” price or leverage existing, broadly deployed platforms (e.g. smart phones, smart home devices, etc).

Automated Systems that use light exposure data: These products/systems/approaches can be informed by any/all of the data gathering systems discussed above, but then used these data directly to take specific actions. These could include:

Lighting systems that adjust lighting conditions in a space (intensity, spectrum, distribution, etc) base at least in part on light exposure information for sensors in associated with the space and/or of people in the space. Lighting systems that adjust to personal exposure data as well as other user inputs such as desired wake and sleep times, sleep tracker data, other tracked health data (such as cognition), or information on an individual’s specific sensitivity to light exposure. Other building systems that can influence light exposure in a space (e.g. automated blinds, automated reflectors on light shelves). Wearable or other personal devices that can influence light exposure on a persons eye (e.g. smart glasses, AR/VR systems, smart contacts).

Data services related to correlations between light exposure and specific health outcomes: If successful establishing a foothold with the products/services above, we will likely build more detailed database on light exposure for people and places that currently exists. These will likely lead to unique and proprietary knowledge related to what types of light exposures have what types of health impacts for what types of people in what types of situations. This information will likely lead to the development of new products/services/patent applications (e.g. if we determine that 10% of the population hyper-sensitive to light exposure, we may develop systems that allow for an easy test for hyper-sensitivity along with products/services that are designed specifically for these users). We many also license or otherwise provide commercial access to our data for researchers or other interested parties to investigate specific light exposure impacts on health.

Value of the SBIR/STTR Project, Expected Outcomes, and Impact. The correct level and spectral content of natural and supplemental lighting that stimulates and supports the circadian rhythm has been consistently shown to reduce sleep disturbances in AD/ADRD patient studies. However, there is currently no practical method of monitoring this “circadian-stimulant” lighting, making it difficult to optimize the patients’ lighting exposure in real-world situations.

With the help of our NIH Phase I and II grants, and the strategic partnerships we have formed, Blue Iris Labs has developed an affordable ambient spectral light exposure-monitoring system which researchers and expert systems can use to monitor circadian lighting metrics continuously and wirelessly, enabling them to apply effective lighting corrections to memory care patients.

Although sleep quality often lies secondary to memory concerns in the public eye, sleep disturbances are one of the most common reasons that families cite for moving their loved ones to care communities and nursing homes. In addition to being a problem for AD/ADRD patients themselves, these sleep disturbances can often lead to sleep problems in AD/ADRD caregivers who must wake up to take care of their AD/ADRD patients. Once the condition has been recognized, there is an answer. In patient studies, it has been shown that effectively managed exposure to direct sunlight, natural interior daylight and other “circadian” lighting consistently reduced sleep disturbances in AD/ADRD. However, there is currently no practical method of monitoring the running exposure levels of circadian lighting, making it difficult to optimize circadian lighting in real-world situations.

The value of our project will be completing the room-based and wearable ambient spectral exposure-monitoring instruments, and app and data-analysis framework, to deliver in-situ data gathering and analysis that will guide patients to effective light therapies. Those therapies can include correct exposure to both natural daylight, as well as supporting other ambient light-exposure adjustments such as tunable, circadian or other “human centric” supplemental light. Ultimately, this will help people with AD/ADRD sleep more soundly. In turn, those patients will enjoy the attendant health benefits of better sleep, as well as better integrate home-care patients to “normal” day/night schedules which enables them to remain in their homes and with their families longer.

The business plan for Blue Iris Labs’ emergence into Phase III requires a task set that includes translation of the data and knowledge acquired in the earlier Phases into actionable recommendations, including apps and other delivery mechanisms to enable rapid adoption beyond the AD/ADRD research community. This essentially amounts to applying the knowledge into real-world use cases. With this SBIR Phase IIB project funding we will expand the Speck room and wearable ambient spectral exposure-monitoring product series from a tool that is suitable for lighting researchers and power users into a tool that provides insights and recommendations for laypeople, through the following efforts:

Implement that “expert recommendation” structure via subscription summary/action reports for institutional setting use and as mobile apps for individual caregivers/patients. Implement the recommendation structure in the context of delivering appropriate data to enable lighting control systems to take the recommended actions. Conduct clinical trials necessary to support commercialization on both systems designed for AD/ADRD care facilities and for people with AD/ADRD who still live at home. Expand our marketing and sales efforts into the AD/ADRD researcher community to drive meaningful initial premium revenue generation. Establish key partnerships and alliances that open faster-track penetration into the AD/ADRD home care and broader health-conscious consumer opportunities

With a foothold in the AD/ADRD researcher and residential care facilities market spaces, we will have both the proof-points and execution capabilities necessary to exercise the leverage with expansion funding partners to enable a high level of growth while still preserving the flexibility to both serve and expand our support of AD/ADRD and cognitive health specialized communities.

Blue Iris Labs’ vision for the future is to be the leading innovator and supplier of Intelligent Spectral IoT modules and big-data infrastructure to help individuals and institutions monitor and control light exposure to enhance health and well-being.

Light Exposure Measurement for ADRD Researchers. Wearable and fixed-location SPD meters for ADRD researchers. Supports real-world study of how light impacts Alzheimer’s disease and related dementia (ADRD) health. Devices that measure SPD and gets these data to the cloud for analysis and/or as a light control input. These are the devices we have developed with NIH funding and are covered at least in part by our prior patent applications.

Light Exposure Measurement for other Health Researchers. Wearable and fixed-location SPD meters for non-ADRD health researchers. Supports real-world study of how light impacts human health generally. Devices that measure SPD and gets these data to the cloud for analysis and/or as a control input. These are the devices we have developed with NIH funding and are covered at least in part by our prior patent applications.

Light Exposure Measurement for building researchers. Wearable and fixed location SPD meters for building researchers. Supports real-world study of how light is used in buildings. Devices that measure SPD and gets these data to the cloud for analysis and/or as a control input. These are the devices we have developed with NIH funding and are covered at least in part by our prior patent applications.

Circadian Control for ADRD facilities. Lighting control systems that utilize SPD inputs for closed loop control in ADRD facility applications. Circadian lighting “solution” that controls the circadian exposure of ADRD patients — using room-based and/or wearable Speck sensors — by automatically adjusting connected lighting systems and/or provide recommendations for patients (e.g. “get more light now” or “get less light now”). Data for control systems could come from other sources as well (e.g. information on target light/spectrum levels based on clinical trials and/or data we have collected from other users). Lighting control systems can access these data (e.g. via the Specks API) and seek to control lighting based on controls algorithms we provide (e.g. match intensity and/or spectrum to predefined targets that vary based on the time of day) or via their own control algorithm (e.g. we don’t know what control signals they are sending to the lights, but we know that the control signals are based at least in part on our sensor data). A system would provide simple, actionable feedback to nursing staff (e.g. a dashboard that shows the actual vs target light exposure for all patients; alerts that can be set if specific patients are below/above specific exposure threshholds, etc.).

Home Circadian control for ADRD. Lighting control systems that utilize SPD inputs for closed loop control in ADRD home care applications. Home-based version of the “Circadian Control for ADRD facilities.” In this case, we may provide a complete “kit” that provides a number of Specks and/or Speck M’s, a gateway, and smart lamps (e.g. Philips Hue or similar) or customized luminaires (e.g. LEDdynamics Perfektlight Table lamp). UI and control algorithms based on ADRD specific use cases.

Home Circadian control for home offices. Lighting control systems that utilize SPD inputs for closed loop control in home office applications. Home office version of “Home Circadian control for ADRD.” In this case, lighting hardware and control algorithms might be more “office” appropriate (e.g. task or ring light rather than table lamp; control based on photopic and glare as well as circadian; control has more user options based on their current needs/tasks).

Fitbit for light. A wearable SPD meter that monitors personal light exposure and provides information and recommendations to the user on how to optimize their light exposure for their health. A consumer wearable version of the researcher-grade wearable. The device allows users a way to track their light exposure much as they can track their steps with other wearables. A companion app can provide recommendations to users based on their exposure and current scientific understanding about the impact of light on health. Apps could include social and/or gamification elements (“your light score was better than 60% of your friends today!”, “congrats! you earned a gold star for hitting your lighting targets for 7 days in a row!” and partnership tie-ins. For example, one could earn points for logging accurate looking data, linking other useful data such as sleep trackers, phone data (location, steps, etc.) Points could be redeemed to get controllable lamps from our partners. Social elements could show users how their light exposure trends compare to the general population or their “friends” group.

Light + Sleep system. A system that includes both a wearable SPD meter and a sleep tracker (wearable or bed-based) that provides information and recommendations to the user on how to optimize their light exposure for their health. A version of the “Fitbit for light” but with the addition of an integration with sleep tracking systems (e.g. Withings mattress, iWatch, Fitbit). Tracking an individual’s light exposure and sleep together will provide very useful data on how light impacts sleep at the population level and at the individual level. At the population level, we should see with increasing resolution what types of light at what times of day most impact sleep. At the individual level, we can see how sensitive the individual’s sleep system is to light exposure and provide customized recommendations. Machine learning would likely be useful for making these correlations and recommendations. If we were successful in this market, there would be a high barrier to entry for competitors because they would need to gather SPD exposure at the population and individual level to do so.

Smart Watch app. An app to be used on existing wearables (e.g. iWatch) that accurately estimates circadian light exposure based on correlations between wrist-based lux measurements and our SPD wearables. This is an app-only option that uses existing smart watches (e.g. iWatch). We have avoided wrist-based measurements because we would rather measure light close to the eye vs at the wrist, and we have avoided commercially available wearables because we would rather measure SPD than just the more limited lux or RGB sensors. But we may be able to build an app that provides a suitable level of accuracy to be useful for circadian light measurements. For example, we might only take light readings when the watch is being viewed by the user (thus placing the sensor in a consistent and more appropriate measurement point that is unlikely to be obscured). Watch data on location, movement, etc. might also provide additional data that could be useful in estimating light exposure (e.g. if a user looks at their watch at the beginning of a run but doesn’t look at it again until they are back inside their house, we can assume that their light exposure during their run was equal to that at the beginning of the run). Further, while we would prefer to have an SPD measurement, circadian stimulus is much more impacted by intensity than spectrum. So we might be able to get at least approximate estimates of CS with just lux readings. And lastly (and perhaps most importantly) we can compare our app-only meter to our (presumably more accurate) SPD measurement approaches (e.g. Fitbit for Light) to validate and/or train the app to increase its accuracy. No other app-only approach could do this (unless they bought a bunch of spectrometers from us or elsewhere).

wearable plus health kit (weight, sleep, exercise). An app that combines circadian light exposure information (from SPD meter or smart watch) with other health data already being collected by a user’s wearable/phone to provide information and recommendations to the user on how to optimize their light exposure for their health. Similar to Fitbit for Light and Smart Watch app but with integration with health data collected by smart phones connected/integrated to the wearable or smart phone, such as Apples Healthkit. This could allow light exposure data to be associated with a variety of health information (weight, sleep, steps, etc.) without the need for further hardware.

Light + photos. A process by which specific SPD measurements can be associated with specific photographs taken by the user to gain a deeper understanding of correlations between SPD exposure and real-world applications. When a user takes a picture at a time and in a location where a wearable device has taken an SPD reading, there maybe be opportunities to associate the SPD of that time/place with the photo. The use cases for these may be varied, ranging from applications that provide a richer photo experience (e.g. when the image is viewed, the lighting in the space adjusts to the SPD that was present when the photo was captured) to mapping the emotional resonance of images/light (e.g. those that we consider beautiful or peaceful or comfortable or even agitated or angry might have a specific SPD signature).

user defined light optimizations (sleep, mood, cognition). An app or process that tracks both SPD exposure and specific user-defined outcomes (better sleep, alertness, improved mood, improved cognition) to provide information and recommendations to the user on how to optimize their light exposure for their desired outcome. We know that light exposure impacts many different biological functions. In many cases, the impacts are likely similar (e.g., if your light exposure is promoting circadian entrainment, it is likely supporting improved sleep, which is in turn supporting many biological functions associated with sleep). But there may be some cases where the outcome that a user might want to optimize are not directly related to sleep (e.g., alertness). Some of biological impacts of light exposure are not well established in part because of the difficulty associated with measuring light exposure. If we can establish statistically significant correlations between light exposure and specific outcomes, we can provide recommendations, etc. that are tailored for these specific outcomes.

Certification of Circadian lighting in offices. SPD monitoring system for offices that allows building managers or 3rd-party partners to certify that circadian lighting targets are being met. A data collection system that estimates the human light exposure in occupied areas of buildings in order to provide 24/7 monitoring and analysis to provide independent 3rd party verification that lighting targets established by the building manager/owner are being met. This is the concept that we have discussed in great detail with UL. We could move forward with this approach with UL, on our own, or with other partners such as the Well Building group.

Verification of Circadian lighting. SPD monitoring system for offices that allows building managers or lighting equipment manufacturers or installers to verify that circadian lighting targets are being met. Similar to Certification of Circadian lighting in offices but this time partnering directly with lighting companies selling tools directly to building managers. In this approach, they won’t have a UL or Well Building certification to point to, but they will have real, 3rd party data that verifies they are providing the light exposure they intend to (or have the information they need to adjust their lighting systems so that they are).

Circadian lighting control in offices. SPD monitoring system that provides SPD (or related) data to lighting control networks to allow for the active (e.g. closed loop) control of lighting systems to achieve circadian lighting targets. A data collection system that estimates the human light exposure in occupied areas of buildings in order to provide 24/7 monitoring and analysis to provide independent 3rd party verification that lighting targets established by the building manager/owner are being met. This is the concept that we have discussed in great detail with UL. We could move forward with this approach with UL, on our own, or with other partners such as the Well Building group.

Certification of SPD in horticulture. SPD monitoring system for offices that allows horticulture/agriculture user or 3rd-party partners to certify that specific SPD-related lighting targets are being met. Similar to Certification of SPD in offices but this time the applications are agriculture and horticulture. It is possible that the current spectral sensor used in the Speck would be appropriate for this application and that the metrics used to evaluate will simply be different calculations than human applications (e.g. PAR as a metric rather than CS). It is also possible that sensors that reach further into the UV or IR may be needed. This application is primarily to provide 3rd-party measurement/verification/certification documenting 24/7 how closely plant light exposure matches user-defined target exposure.

SPD control in horticulture. SPD monitoring system that provides SPD (or related) data to lighting control networks to allow for the active (e.g. closed loop) control of lighting systems to achieve specific SPD lighting targets. Similar to certification for horticulture but now lighting system control is added via API (similar to the control for offices). There are at least two distinct use cases here though: 1) Discovery: growers can use Specks (either as standalone devices or as API-linked parts of a lighting system) to develop/discover specific “light recipes” (what spectrum at what intensity at what duration) to optimize specific targeted outcomes (e.g. total production, fruit growth rates, THC content, promotion/suppression of specific molecules that impact taste, etc.) and 2) Production: Once specific desirable light recipes are discovered, the Specks can be to used verify/control lighting during production to make sure plants receive the target light exposure.

Eye based SPD/CS measurement (Google Glass) algorithm. Measurement and/or control algorithms that are optimized for the measurement of SPD/CS at the eye, designed to leverage the expected emergence of smart wearable glasses which are expected to be networked and to have at least some light sensing systems (which could potentially be calibrated or be correlated to data from our other sensors). This concept is not fully defined but is a recognition that Google, Apple and others have or will soon have Smart Glasses that are networked and have at least some light measurement sensor included. Because of the size and reach of the companies developing these devices, it is likely that they will be able to much more rapidly put networked light sensors in close proximity to users’ retinas than any hardware penetration we might achieve. So, assuming these devices allow 3rd party apps (as Apple and Google currently do for their other devices), we might be well positioned to develop apps for these platforms that leverage our technical and market discoveries to produce apps that have unique value. For example, if these devices do not have a full spectral sensor but rather have a lux or RGB sensor, we might be able to develop apps that correct or convert measurements taken with native sensors to an estimate of SPD or specific circadian metrics. There are several methods we might pursue to achieve these results, such as ground truthing these devices against wearable Specks or even utilizing photograph information along with light sensors data (e.g. the photo can provide information on whether light is coming from the sky or the window or a fire or a lamp). We might also develop apps for these devices based on specific markets we have identified that we either cannot address with our own wearables or we see additional value in also having a glasses-based app (e.g. an app specifically for people with mild cognitive decline that tracks their light exposure and provides circadian health related recommendations). While it is difficult to take this too far without knowing what the technical details of these yet-to-be-released devices may be, it also might be important prepare some potential solutions ahead of time as there might be significant competition here from other similar apps after these devices gain market traction.

SPD data for VR. Detailed database of what SPD people really experience so that makers of VR spaces can more accurately model virtual environments. Virtual Reality (VR) and Augmented Reality (AR) are likely to see significant growth in coming years. A deeper understanding of the lighting conditions that people actually experience in the “real world” (including the SPD they are likely experience in different situations) could be useful for improving the VR and AR experiences. For VR applications, this could involve such things as creating environments that are more realistic renderings of the real world to applications where VR renderings are based not only on providing the visual system stimulus required but also the biological stimulus associated with these environments (e.g. if someone was “virtually” sitting outside in the sun, they would receive light that gives them the visual cues that they are doing this, but they would also receive the circadian exposure that matches this condition). For AR, a deeper understanding of the background lighting conditions in typical applications — including SPD — could help ensure that AR systems appropriately balance brightness/contract/color/etc. between the real world and the augmented material added to it. These ideas primarily describe how AR/VR systems might want to use databases of SPD readings to design better systems. AR systems also potentially directly measure SPD — if they include a spectral sensor — allowing personal SPD exposure to be recorded at the eye and to use these data to inform recommendations and/or automations that seek to improve circadian entrainment and overall health and wellness.

Room Circadian Monitoring with gaze detection. Systems that measure or estimate the gaze of people within a space in order to estimate the SPD/CS they are experiencing based on other data/models that estimate SPD/CS at specific locations and orientations within the space. Camera-based people monitoring systems are getting increasingly sophisticated, with applications that can count the number of people in defined spaces (e.g. to ensure covid19 occupancy limits are being met), track specific individuals, or monitor space utilization. There are a number of ways in which these systems might be used in combination with SPD monitoring systems to provide useful data that might not be possible without each other. For example, a room-based SPD sensor system could include a number of sensors that, together, could estimate the light exposure at an eye-level plane in a room while the camera-based system could determine where people are in the room as well as which direction they (and presumably their eyes) are facing. Folded together, these two datasets could, in theory, provide an accurate estimate of light exposure for each individual in the room without the need for wearable devices.

Calibration app. A calibration app that uses tools on smartphones (GPS, camera, distance measurements) that assists in mapping the locations of all sensors installed in a space to each other and to other items of interest in the space (e.g. lights, windows, the user’s desk or chair). The more we know about the relative locations of sensors and other relavent objects in a space, such as lights, windows, and work areas, the more accurately we can estimate or model the light exposure that people experience in these spaces. This concept is for a system that makes it easier and more accurate to collect these location data. One use case could involve a person (or tripod) standing in the middle of the space being measured and using the phone camera/measurement tools to identify where the sensors are located as they turn 360 degrees. They could also take an SPD measurement in the middle of the room to help improve the model of the room. It may also be possible to provide suggested locations and number of sensors needed to achieve specific SPD/CS measurement features. This could also be used as a sales tool.

live room model. A live model (e.g. map of the CS at the eye-level plane) that is updated based on current sensor data. A graphical representation of space(s) being monitored that includes current measurements of CS or equivalent values as well as estimates of these values at specific locations between sensors (e.g. a point between two sensors could be estimated as an average of these two sensors in the most simple model). These models could be as simple as a 2D plot of the average CS (for all view orientations?) of the eye plane or much more complex models that are 3D, include information on light source and task plane locations, gaze/orientation of occupants, and color/reflectances of room materials. These models can be created using the calibration app or other modeling techniques. Once these models are complete, they can serve as light exposure analytical tools, providing quantitative information showing which areas of the spaces are meeting light exposure targets and which are not, and how these values change throughout the day and throughout the year.

Ground truth modeling techniques (e.g. Speck+iwatch, Speck helping other Specks). A device that includes a device being characterized (e.g. our sensor) and a high accuracy device (e.g. lab grade spectrometer) that continuously collects and analyzes data (perhaps with a neural net) to establish more accurate channel adjustments and measurement algorithms for the device being characterized. This is the device described in the ground truth modeling techniques for calibrating the Specks against spectrometers that have higher accuracy and/or higher resolution.

Groundtruthometer. Techniques that use data from our sensors to increase the accuracy of other SPD/CS measurement systems. iWatches and other similar devices are ubiquitous, and we could potentially reach a much wider customer base if we could develop apps that work on these devices to estimate user SPD/CS exposure, but these systems have at least 2 major limitations: they don’t have sophisticated light sensors and they measure light at the wrist rather than at the eye. If we deploy enough of our wearable sensors in applications alongside wrist-worn wearables, we may be able to establish correlations that allow us to develop improved measurement algorithms for wrist-worn devices. For example, if 10 users wore both Specks and iWatches and these devices took time-synchronized measurements every 5 minutes for 18 hrs a day (not while sleeping) for 10 days, these would yield over 20,000 comparison measurements between SPD/CS and illuminance. We might be able to utilize machine learning or similar techniques to use these data as training data for estimating SPD/CS when only wrist-watch data is collected. If other data is included from the watch (such as if the person is inside or outside, moving or stationary, etc.) we might be able to improve these estimates further. During calibration and/or operation, it may also want to limit wrist-watch measurements so that they only occur when the wrist is in specific locations (e.g., when person moves their arm to look at the watch, triggering the watch screen to activate and for a light reading to be made) so that measurements angles are relatively consistent. If this technique works for improving the accuracy of the watches, it could also potentially be used to increase the accuracy of the Specks themselves (e.g., 10 users could wear a Speck and a device that has higher accuracy and resolution than the Speck for 10 days; or 1 user could do this for 100 days).

FDA approval monitoring device/tool. A measurement kit intended to collect data specifically designed to support FDA compliance for specific health outcomes. There are no lighting systems currently available that have FDA approval as a nonpharmacological treatment for a disease or ailment (except for a few for treating SADD). There are a number of reasons for this but one of them is likely the difficulty in gathering the data needed to document light exposure in applications. Here we would offer a “solution” to manufacturers looking to gather these data. The solution would provide independent 3rd party verification of light exposure and these data could be combined with specific health outcome data (sleep, depression, anxiety, cognition, etc.) measured by others (or potentially by us?) to document causal relationships between light exposure and health outcomes to support clinical trials and/or FDA application documentation.

Kits (home office, ADRD care, at-home plant growers). Easy-to-assemble and use “kits” of lights, sensors, and software for most common applications where measurement and/or control of SPD is valued. Here we envision combining our existing sensors with existing addressable lighting systems to solve specific “problems” including home office lighting, home ADRD care, and agriculture/horticulture needs. These lights could be ones we independently purchase and repackage, but it would be better if we were able to partner with light source companies - both because of the reduction in light source costs and the increase in sales channel support that partnerships would likely yield. A home office kit could include a sensor, wirelessly controllable lamps or tasks lights, and a gateway. The ADRD kit would be similar but might replace the lamps with full-spectrum, circadian table lamps, potentially with a very high total lumen output (both because this is needed for CS and because seniors typically need much more light than younger people for their visual needs). The grower kit would replace the circadian lights with photosynthetically appropriate controllable luminaires/lamps along with a closed loop controller that establishes light exposure targets based on known needs of specific plants (e.g. what do orchids vs basil vs pot plants want and when do they want it?) and delivers what is needed using daylight first and the grow lights as needed to fill in the gaps.

Calibration hallway. An array of sensors assembled to deliver a high level of SPD/CS estimation accuracy within a specific area designed to improve the measurement accuracy of wearable sensors. Users that walk through the “calibration hallway” with their wearables on would have their wearable data “ground truthed” to the highly accurate hallway’s readings. This “hallway” would ideally be an area in a building that has a uniform light distribution (e.g. SPD/CS fairly insensitive to precise location or orientation) and one that is highly trafficked. Fixed location sensors can be carefully installed and calibrated in these applications so that they can record CS/SPD with a high level of accuracy so that they can provide ground truth comparisons to wearables that are passing by. Depending on the measurements, corrections could be made to all the wearable readings (e.g. if all readings for a week are 10% low, the readings from the wearable could be increased by 10%) or just some readings (e.g. if readings for a specific day are off but generally they are not, that day can be flagged as a potentially erroneous application of the wearable). For this scheme to work, a mechanism to determine when the wearable is in “the hallway” would need to be established so that a wearable reading could be triggered and flagged as a comparison point to the hallway readings. One method to do this could be to have a specific beacon or trigger in the hallway for the wearables (e.g., when you are in proximity to this Bluetooth beacon, take a reading, when you see lux readings change in a way that is characteristic of the changes expected when you start to enter the “hallway”, take a spectral reading). Ideally the calibration hallway would have a variety of lights (or different settings?) so that the corrections do not apply only to 1 specific electric light source, though if multiple sources are used, care would need to be made to control uniformity in the space or otherwise ensure the location of the wall sensors and the wearables receive comparable light exposures.

Kickstarter. A marketing campaign aimed at addressing stakeholders in specific stakeholders. This would be a fundraising campaign that seeks to connect with specific communities to bring products/services to the market that get them excited. The most obvious community for this effort would be the AD/ADRD community, where we could leverage our NIH project (and the credibility it offers) to engage the massive ADRD community to help us expand our products/services/solutions related to light exposure and ADRD health to the home care environment. A campaign would aim to make the supporters feel empowered to help advance solutions for a disease they have personally seen the devastating effects of while also providing them with specific benefits based on their donation level (e.g. from free App access for a period of time to a full at home ADRD lighting kit). Similar campaigns could be considered for other communities (from communities associated with other diseases such as cancer, from climate change activists for efficient and healthy home office lighting, etc.).

data used for commissioning / commissioning kit for lighting controls systems. A toolkit specifically designed to help calibrate and commission lighting controls systems. The commissioning of lighting controls systems has long been known as a problem. It typically is labor intensive and not very accurate in real would situations. This leads to unhappy occupants and energy savings that are lower than what is expected. Part of the issue is that the in-situ measurements made during commissioning have inherent limitations. Specifically, as daylight conditions change with different seasons and weather conditions, the original calibration might not be appropriate. A system that can reduce or replace the need for technicians to come to a site multiple times to take measurements and tweak lighting controls settings could fix this. One solution to this problem could involve over-installing a sensor array during initial installation and calibration and then, after some time and with the use of algorithms or automated analysis software, determine which sensors can be safely removed without compromising overall accuracy of measurement and control beyond an accepted threshold. The calibrations sensors could be our standard sensors or the potentially more accurate “groundtruthometer.” Another approach could be a fully independent “calibration sensor system” that places IoT sensors (e.g. the Speck) at critical locations in a space (specific desks, eye level on walls or room dividers that approximate light exposure people typically receive while in these spaces, etc.) that gathers data over a period of time and provides independent recommendations on how the lighting controls systems should be adjusted to meet user-defined goals (e.g. the system is dimming the electric lights too much over at XYZ locations which is resulting in CS levels below target levels - you can fix this by adjusting ABC). Eventually these “corrections” could be automatically communicated to the lighting control system so that adjustments can be made directly, without the need of the user to be involved.

Video conference + circadian lighting solution. A lighting kit designed specifically for the unique visual and biological needs of home workers. A “connected” system of lamps (perhaps including some that are highly circadian-active and some that are minimally circadian active) that are informed by measurements of intensity and spectrum in that space and potentially also information from a wearable (to also use data from when the user was not in the space). The lights could be optimized to provide good “zoom lighting” but also provide good CS and to track and monitor this. This system could target early adopters or other people willing to pay a premium ($2000 for a system?) to have a system that: 1) provides great video conferencing, 2) looks good and works well the rest of the time and 3) provides user feedback or automations on when they need more/less light for their circadian system. This approach leverages the fact that electric lighting systems that are good for video conferencing are also likely to be good for circadian activations (i.e. they both want to shine a bright light in your face).

solutions for the 10 percenters. Systems that are targeted for the people who are especially sensitive to light-based circadian disruption. We often talk broadly about the impact that specific light intensities and spectrum have on the circadian rhythms of people. But the reality is that people have a broad range of sensitivities to light exposure, with some being extremely sensitive to light exposure, some barely impacted by it -it is just the average person at the middle of the bell curve that we are describing when we speak about light exposure and circadian response in general terms. Here, we envision a solution where people can determine if they are among the most light sensitive part of the population (e.g. ideally via a survey or other free or cheap diagnostic tool, but this could also be a lab test or a light/sensor system) and then provide intervention systems that are designed and can be custom-tuned to their specific sensitivities. This is an attractive market because these users are the ones who are most likely to be currently experiencing circadian disruption and they are also the ones most likely to benefit from the solutions we can offer.

Advanced light sensing for Smart Home Applications. A “works with Alexa” or Google Hub spectral light sensor. After much hype and a slow start, the “smart home” seems to finally be taking off and smart lighting is often among the first smart devices to be installed. These systems are increasingly being controlled by voice assistants such as Amazon Alexa or Google Hub for controlling and scheduling lights. Users range from casual consumers who really are just looking for a more convenient way to control their lighting to avid niche users, such as users who integrate color changing lighting systems into their home theatre systems or plant lovers who monitor and control the conditions of their house plants with scientific precision. Missed from these systems are SPD measurements. One potential option to fill this gap would be to provide a “works with Alexa” or similar Speck that could easily be added as an input device for these systems along with a number of software “routines” (e.g. if CS < 0.3, and time between 8 am-2 pm, turn lights up; if sleep score last night was less than 60, then start my transition to night time CS settings 10 mins earlier; closed-loop control for house plant lighting based on PAR sensor readings). Another approach could be to sell complete kits (sensors, lights, and gateway) that can provide specific solutions (as described in the kits section) which are also smart-home-connected.

System to correct or enhance luminaire level illuminance sensors. A system that uses 1 or more Specks and a commissioning tool to “upgrade” the data collected by luminaire based illuminance sensors to provide increased information about light and spectrum in a space. Over the past 5-10 years, the use of luminaire level sensors (or sensors that are built in to luminaires) have become dominant in the market. In many applications, they are now required or encouraged by code. While these sensor systems are easy to install (e.g. it can be as simple as installing the luminaire itself) and can provide a detailed array of information (e.g. illuminance at the ceiling every 8-10 feet in a space), there are significant limitations. The primary limitations are what they are measuring and where they are measuring it. These systems only measure illuminance (not SPD, CS, or even RGB, as far as I am aware) and they measure it at a place people are not. We envision a solution that could connect to the large stock of existing luminaire level lighting systems (at least with networks which are open to allowing 3rd party sensors with credentials to join, which seems to be the direction things are going) that could augment the data being collected at the ceiling plane. One or more Specks in the space could provide important information on the relative distribution of light on the ceiling to those areas of importance in the room itself as well as information on the light spectrum in the room (e.g. the amount and spectrum of natural light vs electric light in specific areas of the room). This information could be useful in creating more accurate estimates of light level and spectrum in the spaces, which could in turn be used to inform control algorithms that attempt to meet specific intensity/spectrum targets in the space.

Speck Sr. A wearable SPD meter that monitors personal light exposure and provides information and recommendations to the user on how to optimize their light exposure to optimize their cognitive health. This is a subcategory of the “fit bit for light” concept where target users are middle-aged or seniors who use (or want to use) apps or other digital tools to track their brain health and take actions to support it. There are apps and other digital health tools that help people track their brain health and/or cognitive decline and it seems likely that these will become more widespread and more sophisticated in the near future. Millions of Americans live at home now with some state of mild cognitive decline. Many/most of these people live relatively normal lives and still use digital devices such as smart phones. In many cases, these emerging digital tools provide quantitative information on the rate of cognitive decline, which can be useful for planning for when they may need more active support and care, or for measuring the effectiveness of particular interventions, such as medications, exercise, or sleep. Additionally, the NIH identifies a small number of factors — with sleep being one of them — that can delay the onset of Alzheimer’s. And the NIH estimates that if the onset of Alzheimer’s is delayed for an individual by 5 years, their life expectancy will increase by 2.7 years (including 5 more non-AD years of life) and the reduction in costs associated with their care would be $500,000.

In this context, we envision a wearable that is designed explicitly for middle-aged and senior users who are interested in pursuing non-pharmacological interventions to reduce their risks associated with mild cognitive decline and AD. (Middle-aged people are included here because the NIH notes that the conditions leading to AD take years to manifest and recommend that people in their 40s do things like exercise, eat well, and sleep well to reduce their risks of getting AD). Such a wearable would monitor the user’s light exposure throughout the day and provide recommendations to the user (via an app and/or other user-friendly UI) on whether they should try to get more or less light at any given moment. The app could also show them when and how much light they should get in the hours ahead so they can plan their activities. This wearable and app light exposure tracker could also be integrated with sleep and/or cognition trackers so that detailed analysis (probably by AI) could compare light exposure to sleep and cognitive data. This may allow for more accurate light exposure recommendations for individuals (e.g. some might be more or less sensitive to light exposure) and would likely provide a gold mine of population level data - particularly if users agree to share other data collected by their phones, including location, user demographics, steps, etc. This system could also integrate with smart home devices so that lights in the house (even those specifically near the wearable) are turned up/down or warmer/cooler based on the current needs of the user. In the case of users with active cognitive decline, algorithms (again, likely AI) would identify the light exposure trends that seem most impactful for that individual and can adjust recommendations accordingly. For all users (in active cognitive decline or not), something similar could happen related to sleep tracking.

We are currently about to start a clinical trial in an AD facility where we are monitoring patient light exposure as well as their cognition, sleep and other factors over several months and with several different lighting conditions. This test is intended to test our ability to accurately characterize the light exposure of patients and document how these changes impact patient health and wellness. This study could help us develop this product and/or help up design and proposed a new, much larger scale clinical trial. If we are able to clinically show even modest links between light exposure and sleep/cognition, the economic benefit could be significant. For example, if we were able to show that a system delays AD onset by just 6 month, the reduction in costs of care would be approximately $50,000. Hard proof of this might not come until we gather a significant amount of data over a long time period (e.g. if we followed demographically similar users in their 50s who have not yet shown signs of cognitive decline and looked at population level sleep/cognition along with their light exposure over months or years). But at the individual level, if users are able to see quantitative proof of how their person light exposure is impacting their own cognition (or sleep), they (and/or their family caregivers) are likely to see value in this.

A business model that might work well in this context one that provides the wearable at a relatively low price (e.g. $99) along with a subscription service (e.g. $10/mo) associated with light exposure tracking, recommendations, and smart-home integrations. If users are seeing quantifiable documentation that their actions related to light exposure are having the intended outcomes for their health, they are more likely to see value in continuing with to pay the subscription month. If/when we have a significant amount of data showing population level improvements in sleep/cognition or delayed onset of AD, it may be useful to investigate an FDA filing for this as a nonpharmacological intervention. This might be necessary down the road to get Medicare reimbursements (Medicare currently pays for approximately 75% of the $300 billion spent annually in the US for AD care). If most individual users or the population data do NOT show positive health outcomes, then we probably shouldn’t be doing any of this as the impact of light exposure is much less expected. This would be a very unexpected result though as prior research by our partners at Mt. Sinai and many others have documented that the impact of light exposure is real and significant. The missing element currently is statistically significant real-world documentation of these impacts.

From an IP perspective, the core concept here is likely a device/system/process for measuring a person’s light exposure (most likely SPD and then any lighting metric that is based on SPD), their sleep, and/or their cognition and providing user-specific recommendations (or automated actions using smart lighting systems) such that recommendations/automations improve AD-related health outcomes.

One concern that I had was that since there are a handful of environmental conditions that one could monitor (e.g. light exposure, temperature, humidity, air quality, etc.) and a handful of AD-related health outcomes one could monitor (e.g. sleep, cognition, aggression, agitation, memory, etc.), perhaps there could already be prior art for the general case. For example, the USPTO probably wouldn’t allow a patent that is as broad as “measure all possible environmental conditions and all possible health outcomes and adjust all possible environmental conditions to optimized all possible health outcomes.” But is there a general case that has been allowed that would make it difficult for us to patent something related to this specific case (e.g. if someone already has a patent or application directly tying light exposure tracking to sleep or cognition tracking)?

Open Source Watch Wearable. A wearable based on existing, open source hardware that includes a light sensor that we can write light exposure tracking software for. For reasons discussed earlier, watches are not ideal for light exposure measurements because they typically do not measure SPD and because their measurements are not always a good proxy for light exposure at the eye. That said, it might be possible to use existing open source smartwatches, such as PineTime, Watchy, Open Source Smartwatch (yes, that’s its name), Culbox, OpenHAK, or Bangle to create a light exposure wearable. Because anything that we do here could be informed by both our expertise in this area (e.g. unique insights about what needs to be measured, when it needs to be measured, what the measurements mean, and what, if anything, the user should be informed about related to their light exposure) and because of the data we will be collecting from our other systems, we may be able to provide more accurate light exposure estimates and more appropriate recommendations to users than competitor smartwatches.

Lighting Genome Project. An initiative to build a public database of SPD data for the greater good of society (while also allowing us to sell a lot of sensors). I’m not sure if we actually want to do this or not but I wrote up the following abstract for a proposed presentation by Mariana and me at LightFair 2022 (June, Las Vegas) and just got word we were selected: “While we now know that light impacts our health in a myriad of ways, hard data on what humans “lighting diet” really looks like is extremely limited. This talk discusses the Lighting Genome Project, a new initiative that aims to gather these data and put them in the public domain. Modelled after the Human Genome Project that mapped the human gene, the Lighting Genome Project will to use new tools (such as wearables and battery powered, networked spectrometers) to gather statistically significant lighting profiles associated with where we work, live, and visit in ways that foster new technologies and approaches that improve the health, quality, and efficiency of the lit environment.”

Data analytics and services. Custom analytics related to light exposure in specific applications. Nearly all of the items discussed in this document will generate data that will add to our growing knowledge and database on the light exposure that people (or plants or specific spaces etc.) experience. As these datasets grow, and as associated information is included, be it demographic or metadata or data collected by other devices (e.g. “these data were collected from a 50 year old man working from home who also tracks his sleep patterns, which are also included here”), they will likely provide, in an ever increasing detail, insights on the impacts on light exposure and the associated social and commercial implications. We expect to use these data ourselves to refine our products and target markets (e.g. we might develop or market products for specific applications where the cause and effect of light exposure tracking and/or interventions are the most pronounced or have the highest economic ROI). But these data could potentially be of great value to others who produce or sell products or services that: 1) can impact light exposure and 2) care about outcomes impacted by light exposure (such as human health). These broadly include: lamps, luminaires, lighting control systems, windows, skylights, agriculture/horticulture, architectural design, healthcare facility design, healthcare practice and procedures, interior furnishings, wall and window treatments, wearables, and many more. Companies in these businesses may see business opportunities in better understanding current light exposure in specific applications, how their products/services could impact (for better or worse) light exposure, and how these impacts on light exposure are likely to translate to the impacts they care about. For example, a window manufacturer might develop a new window film that blocks glare but allows light between 450-500 nm though to maximize circadian impacts. They may wish to purchase data and/or analysis from us that will help them model the light exposure of occupants in class A offices when using standard window films vs their new films. These data could help both in R&D and product development (e.g. identifying how much of a change to light exposure they may need to generate to have a desired impact on human health or other outcomes) and in marketing (through modeling initially and through metering later, they can quantitatively document the expected light exposure benefits their products offer). Ultimately, these data and the correlations and insights they are likely to provide may end up being our most valuable IP.

Speck UV. A Speck sensor that covers the UV light range. In recent years, there has been significant interested in UV lighting. This has primarily been driven by COVID19 concerns, as the use of UV light is known to work well as a viral sterilizer. While UV light has long been known to be potentially hazard to human eyes and skin, many new UV lamps have been installed in buildings, sometimes with limited UV exposure safety considerations. Independent of this, several recent studies have shown that certain spectra and intensities of UV light actually have health benefits, particularly as it relates to vitamin D production. For these reasons, we may wish to develop a Speck UV which is similar to our other Specks but has a UV (or Vis+UV) spectrometer chip rather than a Vis-only chip. This Speck UV could be used to verify that hazardous levels of UV are not reaching areas that humans occupy (e.g. direct or reflected UV from sterilization lamps) or to characterized how UV light exposure impacts human health (such as Vitamin D production) in much the same way our other Specks characterize how visible light exposure impacts circadian health.

Camera AS Spectrometer. A method to approximate SPD based on camera image achieved by gathering substantial training data of simultation camera and Speck measurements.

Camera PLUS Spectrometer. A method that uses a spectrometer (to gather data on composite/average spectrum of a scene) in parallel with a camera (to gather data on color, distribution, and intensity of specific “light rays” in a scene) that allows for a complete mapping of light from the environment to specific points on the retina. There are likely several different methods to estimate SPD at specific locations on a model of a retina using a digital camera and a digital spectrometer. One set of approaches would likely be mathematical in nature (e.g. mapping RGB data from individual pixels of the camera such that they “balance” correctly with the SPD data from the spectrometer) while another set of approaches would likely benefit from machine learning algorithms. A system that gathers these data is thought to be important for a few reasons, including: 1) we are interested in characterizing in the most fundamental, measurable form possible, the “physics” of the physics-to-physiology relationship related to light exposure. A system that could accurately and continuously measure/estimate the angular distribution, intensity, and SPD of light hitting the retina would be this more fundamental data, 2) markets for such as system are probably far off but could include things from research tools for those investigating biological responses to light to AR/VR applications, such as developing systems more accurate and/or “healthy” to systems that are designed for a specific users retinal response (my optometrist took a picture of my retina and emailed it to me on after my last visit...), to robotics applications related to the development of an “artificial eye.”

Herein, a “system” is a group of interacting or interrelated elements that act according to a set of rules to form a unified whole. A “process” is a system in which the elements are actions. Herein, an “expert system” is hardware programmed using artificial intelligence techniques to use databases of expert knowledge to offer advice or make decisions. A light-exposure module is a function that generates brightness and spectral data in the form of estimates or predictions based on one or more inputs, e.g., place, time, and behavior data. “Persistent” means “enduring” and is an antonym of “transitory”. Herein, depending on context, an “application” can be a software program used for productivity such as word processing, data processing, and health services. A “mobile app” is an application running on a mobile device such as a smartphone or smartwatch. Herein, A is “based on” B means B is a factor in determining A. In other words, A is at least a partial function of B.

Herein, “circadian” means “of or pertaining to” natural biological rhythms, especially daily rhythms. “Circadian health” refers to the presence or absence of impairments due to asynchrony with circadian rhythms. “Spectral data” refers to data indicating an amount of light with a subrange of frequencies within the visible light frequency range. Brightness data refers to visible light intensity. Place data is data indicating a place the user may be; place data can be in the form of GPS data, but can be in the form of “indoors” vs (outdoors) or other “type of place” data. Time data is typically absolute or standard data but can begin as relative data that gets time stamped with an absolute time when transferred.

Herein, all art labeled “prior art”, if any, is admitted prior art; all art not labeled “prior art”, if any, is not admitted prior art. The illustrated embodiments, vibrations thereupon, and modifications thereto are provided for by the present invention, the scope of which is defined by the following claims.

What is claimed is:

1. A circadian light-tracking process comprising:
receiving, by a light-exposure model and from a first mobile app, received data, the received data including first place data acquired by a first user device set on which the first mobile app executes; and
estimating, by the light-exposure model, values of spectral parameters based on the place data; and
returning, by the light exposure model to the first mobile app, estimated spectral parameter values.

2. The circadian light-tracking process of claim 1 wherein the light exposure model is included in cloud-based circadian services, the circadian services providing recommendations or instructions regarding circadian light exposure to the first mobile app.

3. The circadian light-tracking process of claim 2 further comprising:
receiving, by the circadian services from a second mobile app running on a second user device set, spectral data and second place data; and
updating the light-exposure model based on spectral and place data received from the second mobile app.

4. The circadian light-tracking process of claim 3 further comprising:
receiving, by the circadian services from a third mobile app running on a third user device set, brightness data and third place data; and
returning, by the light exposure model to the third mobile app, estimated spectral parameter values.

5. The circadian light-tracking process of claim 3 further comprising returning to the third mobile app, by the circadian services, recommendations or instructions regarding circadian light exposure.

6. A circadian light-tracking system comprising:
a first user device set on which a first mobile app executes, the first user device set including a place sensor for acquiring and transmitting first place data regarding locations of the first user device set; and
a circadian services system including a light-exposure model for estimating spectral data based on the first place data and returning the estimated spectral data to the first mobile app.

7. The circadian light-tracking system of claim 6 wherein the circadian services system returns recommendations or automation instructions to the first mobile device based on the estimated spectral data.

8. The circadian light-tracking system of claim 7 further comprising a second user device set on which a second mobile app executes, the second user device including a second place sensor for acquiring and transmitting second place data regarding location of the second user device set and a spectral sensor for providing and transmitting sensed spectral data, the light exposure model being updated based on the updated based on the second place data and the sensed spectra data.

9. The circadian light-tracking system of claim 8 further comprising a third user device set on which a third mobile app executes, the third mobile device set including a brightness sensor for providing brightness data and a third place sensor for providing third place data, the light-exposure model prroviding second spectral estimates based on the brightness data and the third place data, the circadian servicdes system providing the second spectral estimates to the third mobile app.

10. The circadian light-tracking system of claim 9 wherein the circadian services system returns to the third mobile app recommendations or automation instructions based on the second spectral estimates to the third mobile device.

11. Persistent media encoded with code that when executed by a processor implements a process including:
receiving, by a light-exposure model and from a first mobile app, received data, the received data including first place data acquired by a first user device set on which the first mobile app executes; and
estimating, by the light-exposure model, values of spectral parameters based on the place data; and
returning, by the light exposure model to the first mobile app, estimated spectral parameter values.

12. The persistent media of claim 11 wherein the light exposure model is included in cloud-based circadian services, the circadian services providing recommendations or instructions regarding circadian light exposure to the first mobile app.

13. The persistent media of claim 12 wherein the process further includes:
receiving, by the circadian services from a second mobile app running on a second user device set, spectral data and second place data; and
updating the light-exposure model based on spectral and place data received from the second mobile app.

14. The persistent media of claim 13 wherein the process further includes:
receiving, by the circadian services from a third mobile app running on a third user device set, brightness data and third place data; and
returning, by the light exposure model to the third mobile app, estimated spectral parameter values.

15. The persistent media of claim 14 wherein the process further includes returning to the third mobile app, by the circadian services, recommendations, or instructions regarding circadian light exposure.
